(19) United States
(12) Patent Application Publication
(10) Pub. No.: US 2026/0308642 A1
(43) Pub. Date: Oct. 8, 2026

(54) PROCESS FOR CONVERTING RAW BAGASSE INTO PROCESSED BAGASSE AND PROCESSED BAGASSE MADE ACCORDINGLY

(71) Applicant: Chaitanya Ravindra SATHE, Maharashtra (IN)

(72) Inventor: Ravindra Anant SATHE, Pune (IN)

(73) Assignee: Chaitanya Ravindra SATHE, Pune, MH (IN)

(21) Appl. No.: 19/482,346

(22) PCT Filed: Dec. 30, 2023
(86) PCT No.: PCT/IB2023/063406

§ 371 Date: Nov. 7, 2025

(30) Foreign Application Priority Data
Oct. 16, 2023 (IN) 202321070202

(51) Int. Cl. C12P 5/02; D01B 1/14; D01C 1/00

(52) U.S. Cl. CPC C12P 5/023; D01B 1/14; D01C 1/00; C12P 2203/00

(57) ABSTRACT

The present disclosure relates to the process of converting raw bagasse into processed bagasse fiber. In the process a raw bagasse is chopped into pieces. The chopped bagasse pieces are washed with water. The bagasse pieces are soaked and heated in water to remove wax. The bagasse pieces are treated with hydrogen peroxide. The hydrogen peroxide-treated bagasse pieces are again washed. The washed hydrogen peroxide bagasse pieces are treated with alkali. The treated alkali bagasse pieces are dried in a multi-stage drying and moisture is removed. The dried bagasse pieces are pulverized. The pulverized bagasse pieces are sieved to obtain fiber having size up to 707 microns. The processed bagasse can be further used for bio-methanation.

### FIELD

This disclosure relates to a process for converting raw bagasse into processed bagasse and processed bagasse made according to the process.

### Definitions

As used in the present disclosure, the following terms are generally intended to have the meaning as set forth below, except to the extent that the context in which they are used indicates otherwise.

Bagasse: The term bagasse will mean fresh sugarcane bagasse obtained from a sugarcane crushing facility. Typically, such a bagasse will have moisture to the extent of about 60%.

### BACKGROUND

Bagasse obtained from sugarcane crushing contains lignin, cellulosic and hemicellulosic material. Various processes have in tried out for converting this bagasse to make it suitable for biomethanation and into other useful substances. However, because of the toughness of the fiber the bagasse is not useful and is disposed off in landfills. Bagasse is a sustainable product and it has very low impact on an environment.

There is, therefore, felt a need for having a process for converting bagasse into a useful material, particularly suitable for use in biomethanation, as a dietary fiber and also possibly use as an absorbent material.

### Objects

Some of the objects of the present disclosure, which at least one embodiment herein satisfies, are as follows:

An object of the present discloser is to provide a process for converting bagasse into a useful material, particularly suitable for use in biomethanation, as a dietary fiber and also possibly use as an absorbent material.

Another object is to disclose a process that is an economical green, easy to perform, produces a useful material and does not have any byproduct.

Other objects and advantages of the present disclosure will be more apparent from the following description, which is not intended to limit the scope of the present disclosure.

### SUMMARY

The present disclosure relates to a process for converting a raw bagasse into processed bagasse fiber.

In an aspect, the present disclosure relates to a process for converting a raw bagasse into processed fiber. The process comprises a raw bagasse is chopped into a raw bagasse piece. The chopped bagasse pieces are washed with water. The bagasse pieces are soaked and heated in water to remove the wax from bagasse pieces. The bagasse pieces are treated with hydrogen peroxide. The hydrogen peroxide-treated bagasse pieces are washed. The washed hydrogen peroxide bagasse pieces are treated with an alkali. The treated alkali bagasse pieces are dried in a multi-stage drying and moisture is removed. The dried bagasse pieces are pulverized. The pulverized bagasse pieces are sieved. Processed bagasse fiber is obtained having size up to 707 microns.

In an embodiment of the present disclosure, the raw bagasse is taken for processing within 12 to 36 hours after it is received from the sugarcane crushing mill.

In an embodiment of the present disclosure, the bagasse is chopped into pieces in the range of 3 cm to 5 cm.

In an embodiment of the present disclosure, the pieces of chopped raw bagasse is washed with a potable water for about 10 to 25 minutes, the washing is carried out in a main or vessel auxiliary and after washing the washed water is drained.

In an embodiment of the present disclosure, for removing wax from the chopped bagasse the chopped bagasse is soaked in water and the soaked bagasse is heated at a temperature in the range of 55° C. to 65° C., the heated mixture is agitated slowly and after agitation the heated mixture is allowed to stand for 2 hrs and wax is floated on the surface of hot water. The wax is removed by skimming and the hot water is drained out.

In an embodiment of the present disclosure, the water is drained out after the washing or after the removal of the wax.

In an embodiment of the present disclosure, the 2% hydrogen peroxide [commercial grade] is added in a ratio of 1:1.5, with respect to the mass of the chopped bagasse, the bagasse hydrogen peroxide mixture is slowly agitated is to depth the chopped bagasse.

In an embodiment of the present disclosure, the hydrogen peroxide treated bagasse is washed with water with under slow agitation for a period of about 10 minute and the water is drained and is used for making the hydrogen peroxide solution.

In an embodiment of the present disclosure, the hydrogen peroxide-treated washed bagasse is treated with 0.1 N to 0.15 N NaOH, the mass of bagasse to alkali additive was 1:2, the alkali and bagasse mixture is slowly agitated for a period of 30 to 35 minutes and wet washed alkali treated bagasse is obtained by draining out the alkali liquor.

In an embodiment of the present disclosure, multi-stage drying comprises a passing the alkali treated bagasse having moisture in the range of 50% to 60% through a sheet making roll press to obtain a sheet of bagasse having moisture in the range of 25% to 30%; chopping the mat into pieces of 1 cm to 1.5 cm width; passing the bagasse pieces obtained in step b. through a tunnel dryer to obtain partially dried bagasse pieces having moisture in the range of 10% to 15%; and treating the partially dried bagasse pieces obtain in step c. with dried heated air to remove all moisture from the bagasse pieces.

In an embodiment of the present disclosure, the bagasse fibers>707 microns are re-fed to the pulverizer for further grinding to obtain bagasse fibers up to 707 microns for use in bio-methanation.

In an embodiment of the present disclosure, Treated and processed bagasse fibers having size of less than or equal to 707 microns for use in bio-methanation.

### BRIEF DESCRIPTION OF THE ACCOMPANYING DRAWING

The present disclosure will now be described with the help of the accompanying drawing, in which:

FIG. 1 illustrates a schematic system of the process of production of processed bagasse in accordance with an embodiment of the present disclosure;

FIG. 2 illustrates a schematic view showing a laboratory scale apparatus for the purposes of bio-methanation.

### LIST OF REFERENCE NUMERALS USED IN THE DESCRIPTION AND DRAWING

101 feeder
102 dietary fiber
103 rubber clamp
104 inlet tube
104a valve1
105 bio-feed inlet
105a valve2
106 water
107 slurry
108 anaerobic digester
109 water bath
110 thermometer
111 bubbles of gas
112 gas tube
113 water
115 water bath
116 biogas gas
117 measuring cylinder

### DETAILED DESCRIPTION

The present disclosure relates to a system and a method for the production of biomethane gas.

Embodiments, of the present disclosure, will now be described with reference to the accompanying drawing.

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

When an element is referred to as being “mounted on,” “engaged to,” “connected to,” or “coupled to” another element, it may be directly on, engaged, connected or coupled to the other element. As used herein, the term “and/or” includes any and all combinations of one or more of the associated listed elements.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Terms such as “inner,” “outer,” “beneath,” “below,” “lower,” “above,” “upper,” and the like, may be used in the present disclosure to describe relationships between different elements as depicted from the figures.

Bagasse obtained from sugarcane crushing contains lignin, cellulosic, and hemicellulosic material. Various processes have in tried out for converting this bagasse to make it suitable for biomethanation and into other useful substances. However, because of the toughness of the fiber the bagasse is not useful and is disposed off in landfills.

There is, therefore, felt a need for having a process for converting bagasse into a useful material, particularly suitable for use in biomethanation, as a dietary fiber and also possibly use as an absorbent material.

In accordance with one embodiment of this disclosure, the raw but fresh sugar cane bagasse is taken as raw material. Typically, the bagasse is taken for processing within 12 to 36 hours after it is received from the sugarcane crushing mill. Typically, the bagasse that is taken for treatment according to the process as a moisture content between 50 to 60%. This bagasse may be termed as raw bagasse. In one embodiment according to this disclosure, the raw bagasse is chopped by means of a chopper arrangement to pieces of around 3 cm to 5 cm in length.

In one embodiment according to this disclosure, the chopped bagasse is washed with potable water for a predetermined time, typically about 15 minutes for removing extraneous matter.

In one embodiment according to this disclosure, this wash may be carried out in an auxiliary vessel or in the main process vessel.

In one embodiment according to this disclosure, the wash water obtained after washing, with dirt, is drained out from the base outlet of the process vessel.

In one embodiment according to this disclosure, the washed chopped bagasse is soaked in potable water. The drained washed chopped bagasse is covered with water in the process vessel. Water is filled to cover the material completely and the material in the vessel is heated, typically using electrical heating.

In one embodiment according to this disclosure, the material in the process vessel is heated to a temperature in the range of 55° C. to 65° C.

In one embodiment according to this disclosure, the heated material is agitated with the help of an agitator. The typical speed of the agitator is between 10 rpm to 20 rpm. The use of hot water and relatively slow agitation removes wax from the material being processed. The wax floats on the surface. The wax is removed by skimming the surface on the upper part of the vessel. The supernatant liquid is separated using an outlet at the bottom of the vessel.

In one embodiment according to this disclosure, the water drained out from the bottom outlet of the vessel is collected in an effluent tank and further filtered so that it can be used again for one of the steps of the process including the earlier step of soaking the cut bagasse or washing the bagasse.

In one embodiment according to this disclosure, the soaked bagasse is treated with hydrogen peroxide. Typically, the hydrogen peroxide is a solution of 2% hydrogen peroxide [Industrial grade]. The hydrogen peroxide is added to the vessel and the mass is agitated. Typically, the mass is agitated at 20 rpm for a period of 30 minutes to 40 minutes in the vessel. This treatment not only helps in depithing of the chopped bagasse and carbohydrate protection but also oxidizes and further any microbes are totally destroyed because of nascent oxygen. The ratio of bagasse to oxidizing agent is maintained at a pre-determined ratio typically 1:1.5. Instead of hydrogen peroxide any other suitable oxidizing agent can be used.

Following the treatment with hydrogen peroxide, the liquor is removed from the process vessel and a freshwater wash is given to the remaining mass with an agitation speed of 10 rpm to 20 rpm for a period of 10 minutes. The wash liquid is also drained using the bottom outlet.

The washed mass is treated in the vessel with 0.1 N to 0.15 N of NaOH in a typical ratio of mass to alkali additive will be 1:2. Instead of NaOH any other suitable alkali may also be used. Typically, the treatment with alkali is done under agitation, typically at a speed of 20 rpm for a period of 30 to 35 minutes. After alkali treatment, the liquor is drained out and the wet mass is taken out of the vessel. The wet mass is approximately 50% to 60%.

In one embodiment according to this disclosure, the wet mass is passed through a sheet-making roll press, typically with a load of 800 kg to 1000 kg.

In one embodiment according to this disclosure, the treatment in the roll press helps in removing moisture percent in the range of 25 to 30%.

In one embodiment according to this disclosure, from the roll press, a sheet is formed which is cut to a suitable size. Typically, the mat is cut into pieces of 1 cm to 1.5 cm in width. These cut pieces of treated bagasse mat have a moisture.

In one embodiment according to this disclosure, the moisture deficient cut pieces are passed through a dryer. Typically, the dryer is a tunnel drier. The cut pieces are passed using conveyor belts so that the pieces are exposed to dry heat having moisture 10% to 15%.

In one embodiment according to this disclosure, dehumidified heated air is used typically, at a temperature of 55° C. to 60° C. which is consistently maintained. This step takes out some more moisture and finally the treated bagasse pieces has 0% to 1% of moisture in it.

In one embodiment according to this disclosure, the output processed bagasse from drier is fed to a micro-pulveriser in which using various sieves particulate matter of different sizes are obtained which can be used for different purposes.

In one embodiment according to this disclosure, the sieves configured to serve processes fiber up to 707 microns. The sieved product can be used as follows:
1. Up to 707 microns particle size for bio-methanation.

All contact surfaces are of food-grade steel.

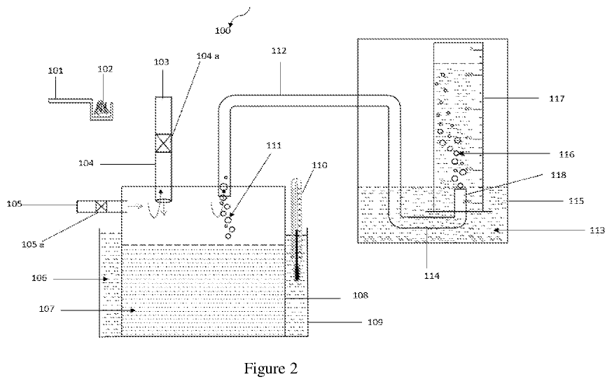

In the present embodiment, the present disclosure provides a laboratory system for the production of biogas gas; shows in FIG. 2.

Referring to FIG. 2, the system for (100) comprises at least one feeder (101), at least fibrous material (102), at least one inlet tube (104) consisting of a (104a) valve 1, at least one rubber clamp (103), at least one water bath (109) consist water (106) and one thermometer (110) below the at least one anaerobic digester (108), an anaerobic digester comprises a slurry (107), at least one gas tube (112) consist a bubble of gas (111), at least one water bath2 (115) consist a water (113), at least one outlet gas channel, at least biomethane gas (116), at least one measuring cylinder (117).

In an another aspect of the present disclosure, the fibrous material (102) is the raw material for the process and is inserted it in an inlet tube (104) by opening a valve (104a) through a feeder (101).

In an another embodiment of the present disclosure, a cattle dung is passed through a bio-feed inlet (105) by opening a valve (105a).

In an another embodiment of the present disclosure, both fibrous material and cattle dung is passed in an anaerobic digester (108). A slurry is formed by the mixing of cattle dung with water and fibrous material in the anaerobic digester.

In an another embodiment of the present disclosure, a water bath1 (109) is placed below the anaerobic digester.

In an another embodiment of the present disclosure, after mixing all the slurry the bubbles (111) are passed through the gas tube (112) and it is connected to the measuring cylinder (117) and placed in the water bath 2 (115).

In an another embodiment of the present disclosure, a reaction occurs at 39° C. temperature which is recorded by the thermometer (110) present in water bath1 (109), and the bubble of biogas containing methane (111) gas is transferred through the outlet gas channel (118) and collected in a tube.

In an another embodiment of the present disclosure, the processed bagasse fiber introduced through the inlet tube falls on the surface of the slurry. Initially, they float but they also start absorbing water from the slurry and act as super-absorbents. The fibers are able to absorb water at over five times their own mass. As they become heavy as a result of the water absorbed; the fiber particles sink into the slurry and suspend themselves at different levels in the slurry. These water-filled fiber particles act as nutrients for the microbes in the slurry by the process of anaerobic digestion releasing gas which includes methane.

The lab apparatus may be scaled up for use in a pilot plant or a commercial bio-methanation plant.

Invention is now be described through the following example and experiments but examples and experiments are only illustrative and do not in any way restrict the disclosure of the invention.

The fiber produced was tested on a cow dung gas production plant set-up. Average cow dung gas production was 40 liter/kg. The initial gas volume produced is not measured for the first four days. Thereafter, the liquid was used further as an inoculant and experiments and trials were conducted as mentioned below. After the 5th day, the slurry gets exhausted from gas. All experiments are carried out after the 5th day.

### Example 1

500 μm of a raw bagasse having a moisture of about 50% was taken for the process. The raw bagasse was chopped into small pieces about 30 to 50 mm length. Further, the chopped bagasse was washed with water for 15 min. The chopped bagasse in the process vessel was heated to a temperature of 60 degrees. The heated bagasse was agitated with the help of an agitator rotated at 20 rpm. After likely the heated bagasse was allowed to stand for 2 hrs. The wax separated from the bagasse floated on the surface of the water, and by skimming the surface the wax was removed, and the excess water was drained from the bottom outlet of the vessel to obtain the wax-deficient soaked bagasse.

The wax-deficient soaked bagasse was again placed in the process vessel and was treated with 750 ml of 2% hydrogen peroxide and agitated in the process vessel at 20 rpm for 30 minutes to obtain an oxidized bagasse. Freshwater was added to wash the oxidized bagasse. For washing, the vessel was agitated at a speed of 10 rpm for 10 minutes to obtain washed bagasse. The excess water was drained from the vessel. The washed bagasse was then treated with 1 liter of 0.1 N NaOH and agitated at a speed of 20 rpm for 30 minutes to obtain alkali-treated bagasse. After alkali treatment, the liquor was drained out to obtain alkali-treated wet bagasse mass. The alkali-treated wet bagasse mass was then passed through a sheet-making roll press with a load of 800 kg to remove moisture and obtain a bagasse sheet. The obtained bagasse sheet was chopped to a suitable size such as 1 mm. The chopped pieces had a moisture of about 25%. The chopped pieces were passed through a tunnel dryer, where heated air at a temperature of 55° C. was used. The dehumidified bagasse pieces had a moisture of about 10%. The dehumidified bagasse pieces were further dried with hot air and pulverized using a micro-pulverizer to obtain pulverized bagasse.

The pulverized bagasse was sieved by using a sieve to obtain the bagasse fiber up to 707 microns and further taken up for bio-methanation stage 2.

### Experiment 1: The Biogas Production in the Absence of Bagasse Fiber:

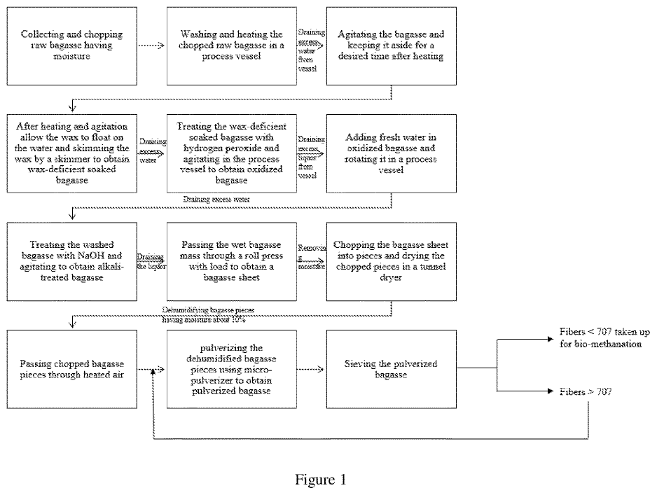

500 μm of cattle dung was added into 3 liters of water to prepare a slurry. The slurry was prepared in an anaerobic digester that had a capacity of 5 liters. The digester was connected after 72 hours to the apparatus for collecting biogas as seen in FIG. 1) and the yield of biogas every 24 hrs was noted and the results were tabulated in Table (1) below:

TABLE 1

| Number | Amount of biogas | Content of methane in |
|---|---|---|
| of Days | collected | the biogas |
| Day 1 | 42 ml | 8% |
| Day 2 | 41 ml | 9% |
| Day 3 | 40 ml | 10% |
| Day 4 | 43 ml | 7% |
| Day 5 | 42 ml | 8% |
| Day 6 | 40 ml | 8% |
| Day 7 | 41 ml | 9% |
| Day 8 | 44 ml | 9% |
| Day 9 | 45 ml | 10% |
| Day 10 | 45 ml | 7% |

### Experiment 2: The Biogas Production by Using Feed of 707-Micron Size of Bagasse Fiber:

500 μm of cattle dung was added into 3 liters of water to prepare a slurry, and initially was exhausted for three days and then was added to the digester having capacity of 5 litres. [The slurry was prepared in an anaerobic digester that had a capacity of 5 liters]. 1 μm of bagasse fiber having particle size up to 707 micron was added every day for 24 days and then feeding was stopped for the next 5 days and started again. The digester was connected to the apparatus for collecting biogas as seen in FIG. 1) and the yield of biogas every 24 hrs was noted and the results were tabulated in Table (2) below:

TABLE 2

| Number | Amount of biogas | Content of methane in |
|---|---|---|
| of Days | collected | the biogas |
| Day 1 | 500 ml | 57% |
| Day 2 | 520 ml | 58% |
| Day 3 | 550 ml | 59% |
| Day 4 | 560 ml | 55% |
| Day 5 | 580 ml | 54% |
| Day 6 | 520 ml | 53% |
| Day 7 | 550 ml | 54% |
| Day 8 | 560 ml | 55% |
| Day 9 | 560 ml | 60% |
| Day 10 | 510 ml | 61% |
| Day 11 | 520 ml | 65% |
| Day 12 | 530 ml | 63% |
| Day 13 | 570 ml | 62% |
| Day 14 | 550 ml | 60% |
| Day 15 | 520 ml | 60% |
| Day 16 | 540 ml | 65% |
| Day 17 | 550 ml | 66% |
| Day 18 | 560 ml | 65% |
| Day 19 | 550 ml | 60% |
| Day 20 | 550 ml | 53% |
| Day 21 | 540 ml | 52% |
| Day 22 | 520 ml | 51% |
| Day 23 | 510 ml | 51% |
| Day 24 | 500 ml | 50% |
| Day 25 | 430 ml | 8% |
| Day 26 | 410 ml | 9% |
| Day 27 | 400 ml | 10% |
| Day 28 | 410 ml | 7% |
| Day 29 | 420 ml | 8% |
| Day 30 | 410 ml | 8% |
| Day 31 | 560 ml | 66% |
| Day 32 | 570 ml | 67% |
| Day 33 | 570 ml | 67% |
| Day 34 | 590 ml | 68% |
| Day 35 | 590 ml | 68% |

### Experiment 3: The Biogas Production by Using a Size Above 707-Micron of Bagasse Fiber:

500 μm of cattle dung was added to 3 liters of water to prepare a slurry, and initially was exhausted for three days and then was added to the digester having capacity of 5 litres. The slurry was prepared in an anaerobic digester that had a capacity of 5 liters. 1 μm of bagasse fiber was added every day having particle sizes above 707 microns. The digester was connected to the apparatus for collecting biogas as seen in FIG. 1) and the yield of biogas in 24 hrs was noted and results were tabulated in Table (3) below:

TABLE 3

| Number | Amount of biogas | Content of methane |
|---|---|---|
| of Days | collected | in the biogas |
| Day 1 | 450 ml | 18% |
| Day 2 | 430 ml | 17% |
| Day 3 | 420 ml | 19% |
| Day 4 | 480 ml | 20% |
| Day 5 | 450 ml | 20% |
| Day 6 | 470 ml | 19% |
| Day 7 | 440 ml | 18% |
| Day 8 | 430 ml | 17% |
| Day 9 | 420 ml | 20% |
| Day 10 | 460 ml | 19% |

### Experiment 4: Using Conventional Chopped Bagasse for Bio-Methanation:

500 μm of cattle dung was added to 3 liters of water to prepare a slurry, and initially was exhausted for three days and then was added to the digester having capacity of 5 litres. [The slurry was prepared in an anaerobic digester that had a capacity of 5 liters]. Unprocessed bagasse fiber was pulverized through the pulverizer to obtain fiber less than 707 microns. 1 μm of unprocessed bagasse fiber was added every day. The anaerobic digester was connected to the apparatus for collecting biogas as seen in FIG. 1) and the yield of the gas in 24 hrs was noted and results were tabulated in Table (4) below:

TABLE 4

| Number | Amount of biogas | Content of methane |
|---|---|---|
| of Days | collected | in the biogas |
| Day 1 | 150 ml | 40% |
| Day 2 | 159 ml | 45% |
| Day 3 | 160 ml | 49% |
| Day 4 | 160 ml | 50% |
| Day 5 | 140 ml | 50% |
| Day 6 | 150 ml | 45% |
| Day 7 | 158 ml | 48% |
| Day 8 | 140 ml | 49% |
| Day 9 | 150 ml | 50% |
| Day 10 | 160 ml | 46% |

Conclusion: Therefore it can be concluded that processed bagasse fiber below 707 microns according to this method of disclosure is best suitable for use as a feed for bio-methanation.

### Technical Advancements

The present disclosure described herein above has several technical advantages including, but not limited to, the realization of a process for converting bagasse into processed bagasse, that:
can be suitable for use in biomethanation, as a dietary fiber;
can be use as an absorbent material; and
is an economical green process for producing processed bagasse, easy to perform, produces a useful material and does not have any byproduct; and
can be used as a dietary fiber, as an absorbent or for bio-methanation.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A process for converting a raw bagasse into processed bagasse fiber, said process comprising a following step:
i. chopping the raw bagasse into raw bagasse pieces;
ii. washing the chopped bagasse pieces;
iii. soaking and heating the washed bagasse pieces to remove wax from the bagasse pieces;
iv. treating the bagasse pieces with hydrogen peroxide;
v. washing the hydrogen peroxide treated bagasse pieces;
vi. treating the washed hydrogen peroxide treated bagasse pieces with alkali;
vii. multi-stage drying of the alkali treated bagasse pieces to remove the moisture from the bagasse pieces and obtained dried treated bagasse pieces; and
viii. pulverizing the dried bagasse pieces and sieving the pulverized bagasse pieces to obtain processed bagasse fiber having size up to 707 microns;

2. The process as claimed in claim 1, in which the raw bagasse is taken for processing within 12 to 36 hours after it is received from the sugarcane crushing mill.

3. The process as claimed in claim 1, in which in step (i) the bagasse is chopped to pieces of 3 cm to 5 cm.

4. The process as claimed in claim 1, in which wherein in step (ii) washing is carried out with potable water for about 10 to 25 minutes, the washing is carried out in a main or vessel auxiliary and after washing the washed water is drained.

5. The process as claimed in claim 1, in which in step (iii) for removing wax from the chopped bagasse the chopped bagasse is soaked in water and the soaked bagasse is heated in the temperature in the range of 55° C. to 65° C., the heated mixture is agitated slowly and after agitation the heated mixture is allowed to stand to unable wax to float on the surface of the hot water, the wax is removed by skimming and the hot water is drained out.

6. The process as claimed in claim 5, in which the water drained out in step (iii) is reused after filtration in either step (i) and/or step (iii) of the process.

7. The process as claimed in claim 1, in which in step (iv) the hydrogen peroxide is a 2% solution of hydrogen peroxide, and is added in a ratio of 1:1.5, with respect to the mass of the chopped bagasse, the bagasse hydrogen peroxide mixture is slowly agitating is to de-pith the chopped bagasse.

8. The process as claimed in claim 1, in which in step (v) the hydrogen peroxide treated bagasse is washed with water with under slow agitation for a period of about 10 minute and the water is drained and is used for making the hydrogen peroxide solution.

9. The process as claimed in claim 1, in which in step (vi) in which the hydrogen peroxide treated washed bagasse is treated with 0.1 N to 0.15 N NaOH, the mass of bagasse to alkali additive is 1:2, the alkali and bagasse mixture is slowly agitated for a period of 30 to 35 minutes and wet washed alkali treated bagasse is obtained by draining out the alkali liquor.

10. The process as claimed in claim 1, in which in step (vii) the multi-stage drying comprises:
a. passing the alkali treated bagasse having moisture in the range of 50% to 60% through a sheet making roll press to obtain a sheet of bagasse having moisture in the range of 25% to 30%;
b. chopping the mat into pieces of 1 cm to 1.5 cm width;
c. passing the bagasse pieces obtained in step (b). through a tunnel dryer to obtain partially dried bagasse pieces having moisture in the range of 10% to 15%; and
d. treating the partially dried bagasse pieces obtain in step (c). with dry heated air to remove all moisture from the bagasse pieces.

11. The process as claimed in claim 1, in which in step (viii) the bagasse fibers>707 microns are re-fed to the pulverizer for further grinding to obtain bagasse fibers up to 707 microns for use in bio-methanation.

12. Treated and processed bagasse fibers having size of less than or equal to 707 microns for use in bio-methanation.
